(19) United States
(12) Patent Application Publication
(10) Pub. No.: US 2009/0292235 A1
(43) Pub. Date: Nov. 26, 2009

(54) Cardiotomy Reservoir with Total Massive Air Emboli Protection Safety Valve

(76) Inventor: Vishwas Kiran Paul, Maharashtra (IN)

Correspondence Address: NOTARO & MICHALOS P.C., 100 DUTCH HILL ROAD, SUITE 110, ORANGEBURG, NY, 10962-2100, US

(21) Appl. No.: 11/909,272

(22) PCT Filed: Aug. 3, 2005
(86) PCT No.: PCT/IN05/00255

§ 371 Date: Sep. 21, 2007

(30) Foreign Application Priority Data
Mar. 24, 2005 (IN) 331/MUM/2005

(51) Int. Cl. A61M 1/14; A61M 39/22

(52) U.S. Cl. 604/6.16; 604/256

(57) ABSTRACT

A total massive air emboli protection safety valve during cardio pulmonary bypass to protect the massive air embolism without allowing any air into the arterial system by keeping a minimum level of blood into venous cordiatomy reservoir, keeping the patient's life very much safe. It will be in-built in the resent hard shell cordiatomy reservoirs and no need to attach and pay anything extra.

The present invention relates to a total massive air emboli protection safety valve during cardio pulmonary bypass (CPB). The said invention is directed to provide an instrument, which could help and eliminate the life-threatening problems, which occur frequently due to the massive air embolism. The said invention is developed mostly for use in Cardio pulmonary bypass (CPB) process.

The present invention is based on the nature's law of floating substances over the fluid and aims to bring down the dangerous and life threatening cases, which takes place during Cardio pulmonary bypass (CPB) due to massive air entry into the aorta leading to neurological complications like unconsciousness, paraplegia, decortications, de-cerebration and even brain death. The object of the invention is to protect, prevent and save the patients from the sudden mishaps, which could occur due to massive air entry into the aorta while undergoing Cardio pulmonary bypass (CPB).

The purpose of the invention is to protect the massive air embolism during Cardio pulmonary bypass (CPB) by preventing the air to pass into the arterial system, which is attained by maintaining minimum level of blood into the venous cordiatomy reservoir and thereby keeping the patients life out of danger.

The present invention prevents the venous reservoir to run-dry due to insufficient venous return caused by accidental incidence. The present invention is used in cardiac centre for protecting the massive air emboli without emptying the fluid and also not allowing any air after the cordiatomy and thereby totally eliminating the risk of massive air emboli.

The scope of the present invention is to protect the air entry, if any in the system, without doing any refilling of fluids and disconnecting the arterial system tubing's. The valve is inbuilt into resent hard shell cardiotomy reservoirs and does not require any extra attachments or cost. In case of accidental stoppage of venous blood and the re-bypass can be established within few seconds whenever we will get sufficient venous return without doing any de airing or disconnection of circuit.

### PRIOR ART

In the prior art, the design, construction and mechanism is of such a nature that they do not really provide to protect and prevent the total massive air entry into the aorta during Cardio pulmonary bypass (CPB). There are four types of devices available at present, which are used during Cardio pulmonary bypass (CPB) to protect and prevent the massive air entry. They are as follows:
a) Electronic level sensors.
b) Biomedicus pump.
c) Arterial Filters and
d) Collapsible reservoirs
   All these above mentioned devices have failed to provide the function of protecting and preventing the total massive air entry during Cardio pulmonary bypass (CPB).

The electronic level sensor is attached to the outlet part of the cardiotomy reservoir to sense the cardiotomy blood level. Whenever the blood level falls below the specified level, the infrared sensor blows the alarm. In case due to some unforeseen circumstances or any technical defects the infra-red sensor fails to perform, the complications during the operation are bound to arise.

The biomedicus pump in place of arterial pump is attached to the cardiotamy reservoir through ⅜ tubing. It is used to pump the blood into aorta and for uniform supply of blood in the human body. If the cardiotamy becomes empty due to lack of venous blood return to cardiotamy, it will pump the massive air into the heart and creates complications.

Arterial filters which are attached in arterial line after the oxygenators and near to the heart to remove micro air bubbles through its filters. In case the massive air comes during the operation, the air filter becomes empty and filled with air, which is further pumped into the heart and creates complications.

Collapsible Reservoir bags are attached to the outlet of cardiatomy reservoir to protect the massive air embolism when the cardiatomy reservoir gets empty. But due to vigorous suction pressure of the arterial pump the collapsible reservoir bags fails to perform its function efficiently and to its optimum capacity and therefore allows well extent of air in the aorta/heart and creates complications.

### DEFICIENCIES OF PRIOR ART

1. The Electronic level sensor cannot be substituted to proper vigilance due to manufacturing errors, loose connections, operating errors & breakage in sensor cables & components resulting in malfunctioning of the sensors which further creates problem of massive air embolism.
2. That the Electronic level sensor requires heavy investment and expenditure.
3. The Arterial line filters & the biomedicus pumps are capable to remove micro air embolism and are incapable to protect the creation of massive air embolism.
4. That the Arterial line filters & biomedicus pumps requires high cost of expenditure, as they need to dispose off at regular intervals.
5. The Collapsible Reservoir bags, which are attached after the cordiatomy reservoir does not protect massive air embolism totally. It allows well extent of air in arterial system after the cordiatomy reservoir becoming empty due to vigorous suction pressure of the arterial pump head.

### PRESENT INVENTION

With a view to overcome the deficiencies found in the prior art, the present invention is introduced. According to present invention a total massive air emboli protection safety valve during cardio pulmonary bypass to protect the massive air embolism without allowing any air into the arterial system keeping a minimum level of blood into venous cordiatomy reservoir, keeping the patient's life very much safe. It will be in built in the resent hard shell cordiatomy reservoirs and no need to attach and pay anything extra.

The present invention is an in built cordiatomy reservoir. A small cylindrical tube is fixed inside over ⅜ outlet of the cordiatomy reservoir. The cylindrical tube has holes exactly opposite to each other from the inside bottom level of the outlet port for uniform flow of blood through the holes. The cylindrical tube contains a piston stem with a rubber cock at the bottom of the stem. An airtight container is fixed at the top of the piston stem, which moves freely in the up and down direction inside the cylindrical tube. The piston slippage pins are placed to protect the slipping of piston from the cylindrical tube and further enables to carry on the activity of bypass safely and without taking any risk.

Therefore according to the present invention A total massive air emboli protection safety valve for use during cardio pulmonary bypass to protect the total massive air embolism, comprising of a small cylindrical tube fixed inside over the outlet of cardiatomy reservoir, and is connected to the pre-pump head line; this cardiatomy reservoir is filled with blood fluids when the pump is on to perform the Cardio Pulmonary Bypass, the said cylindrical tube has holes opposite to each other from the inside bottom level of the Cardiotomy reservoir outlet port, for uniform flow of blood through the holes; further the said cylindrical tube contains piston stem with a rubber cock at the bottom of the piston stem; an airtight container is also fixed at the top of the piston, which moves freely in up and down direction inside the cylindrical tube; the piston slippage pins are placed at the top of cylindrical tube to protect the slipping of piston from the cylindrical tube; the valve and the rubber cock in the cylindrical tube starts floating over the fluid and the blood starts to flow through the side holes of cylindrical tube towards the outlet opening of the reservoir and pre-pump head tubing; as soon as the venous return is stopped due to any reason and the reservoir level drops, the floating valve moves down and gets stuck on the inside opening of the cardiotomy reservoir to keep minimum level of blood in the reservoir and to assure that it never gets empty, thereby protecting the air to enter into the system; on receipt of sufficient venous return and blood level in the reservoir, the sufficient fluid level releases the safety valve and rotates the pump in reverse motion for few rotation only to float again in the fluid and to slowly stop and increase the pump flow in the right direction and thereby re-establishing safety in Cardio Pulmonary By-pass within few seconds, which continue to provide sufficient fluid level and keeps the patient's life out of danger.

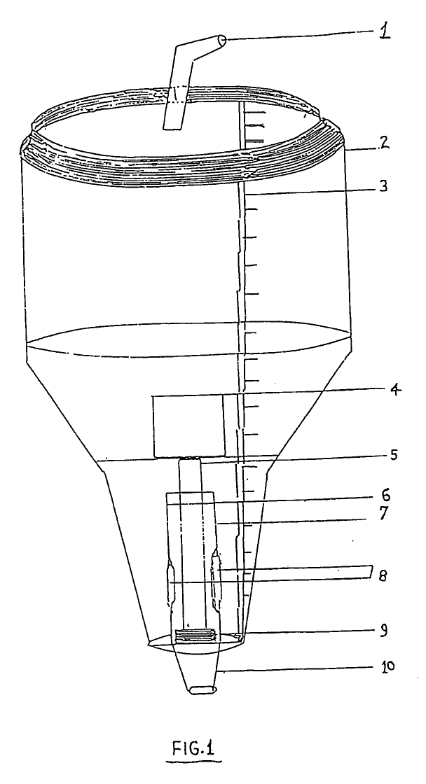

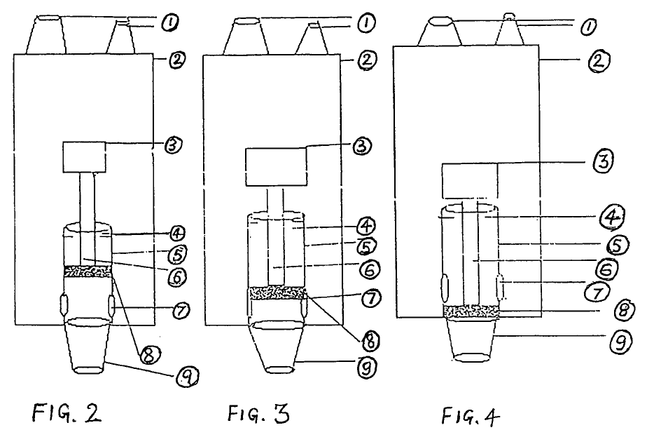

The invention is now described with the help of accompanying drawing i.e. FIG. 1, FIG. 2, FIG. 3 and FIG. 4 wherein FIG. 1 shows the construction of the safety valve in a cordiatomy reservoir. FIG. 2 shows that the safety valve is floating due to high blood level in the cordiatomy reservoir. FIG. 3 shows the reduction in blood level, resulting in the movement of safety valve towards outlet opening according to fluid level. FIG. 4 shows that further reduction in the blood level results in blocking the outlet opening by safety valve.

FIG. 1 shows the construction of the safety valve comprising of a fluid inlet (1) through which the fluid in cardiatomy reservoir is filled; a small cylindrical tube (7) is fixed inside over the 3/8 outlet port (10) of the cordiatomy reservoir (2). The cylindrical tube (7) has holes exactly opposite to each other from the inside bottom level of the Cordiatomy reservoir outlet port (10) for uniform flow of blood through the holes (8). The cylindrical tube (7) contains a piston stem (5) with a rubber cock (9) at the bottom of the piston stem. An airtight container (4) is fixed at the top of the piston (5), which moves freely in up and down direction, inside the cylindrical tube. The piston slippage pins (6) are placed to protect the slipping of piston (5) from the cylindrical tube (7).

FIG. 2 shows that during Cardio Pulmonary Bypass (CPB), when the blood level is high, the safety valve floats. During the period, when the blood level is high, there are no chances of massive air embolism due to the reason that there is sufficient amount of blood available in the cordiatomy reservoir to perform the Cardio Pulmonary Bypass (CPB) and therefore life of the patient is out of danger.

FIG. 3 shows that there is direct connection between the blood level and the safety valve. It shows that reduction in the blood level proportionately brings down the valve towards outlet opening and will lead towards outlet opening, to block the same.

FIG. 4 shows that due to sudden reduction in blood level, the blood level has now reached a stage where there is no further reduction in blood level is affordable due to safety valve during Cardio Pulmonary-Bypass (CPB). We have seen in FIG. 3 that the safety valve is moving towards outlet opening. Due to sudden reduction in the blood level the valve will continue to move towards the outlet opening and will get stuck on it to prevent total massive air emboli and thereby saves the precious life of the patient and prevent from other major life threatening problems.

### ADVANTAGES OF THE INVENTION

1. That the safety valve protects and prevents the flow of air into the arterial system.
2. That the said invention is 100% safe and easy to re-establish the CPB within few seconds, whenever we get sufficient fluid level.
3. That there is no need to remove the air manually from the oxygenator and the arterial tubing's.
4. That the said invention is effective and efficient as it saves lot of time in re-establishing the CPB.
5. To provide additional attachments and adding of fluids can be avoided with this invention.
6. That the said invention provides comfort and ease while conducting the CPB. The said CPB can also be conducted without any fear and risk.
7. The life of the patient is totally secured, as there is no chance of Massive Air Emboli.
8. The said invention is flexible in nature as it can also be used in any other hollow chamber and fluid reservoir to prevent air entries in the tubing's and applicable fields.

1. A total massive air emboli protection safety valve for use during cardio pulmonary bypass comprising of a small cylindrical tube fixed inside over the outlet port of the cordiatomy reservoir; the said cylindrical tube has holes opposite to each other from the inside bottom level of the Cordiatomy reservoir outlet port for uniform flow of blood through the holes; further the said cylindrical tube contains piston stem with a rubber cock at the bottom of the piston stem; an airtight container is also fixed at the top of the piston, which moves freely in up and down direction inside the cylindrical tube; the piston slippage pins are placed at the top of cylindrical tube to protect the slipping of piston from the cylindrical tube.

2. A total massive air emboli protection safety valve as claimed in claim 1, when used during cardio pulmonary bypass to protect the total massive air embolism, comprising of a cardiatomy reservoir outlet, which is connected to the pre-pump head line and is filled with blood fluids; when the pump is on to perform the Cardio Pulmonary Bypass, the valve and the rubber cock in the cylindrical tube starts floating over the fluid and the blood starts to flow through the cylindrical tube side holes towards the outlet opening of the reservoir and towards pre-pump head tubing; as soon as the venous return stops and the reservoir level drops due to any reason, the floating valve moves down and gets stuck on the inlet opening of the cardiotomy reservoir to keep minimum level of blood in the reservoir and ensures that it never gets empty to protect the air entry into the system; on receipt of sufficient venous return and blood level in the reservoir, the sufficient fluid level releases the safety valve and rotates the pump in reverse motion for few rotation only, to float again in the fluid and to slowly stop and increase the pump flow in the right direction and thereby re-establishing safety in Cardio Pulmonary By-pass within few seconds, which continue to provide sufficient fluid level and keeps the patient's life out of danger.

3. A total massive air emboli protection safety valve substantially as herein described and illustrated in the accompanying drawings.
